(19) United States
(12) Patent Application Publication
(10) Pub. No.: US 2014/0193364 A1
(43) Pub. Date: Jul. 10, 2014

(54) TAM RECEPTOR LIGANDS, METABOLITES AND PRECURSORS THEREOF IN THE DETECTION AND MODULATION OF INFLAMMATORY NEUROPATHOLOGICAL DISEASE

(71) Applicants: Trevor Kilpatrick, Parkville (AU); Michele Denise Binder, Skye (AU); Zhi-Ming Gerry Ma, Bulleen (AU)

(72) Inventors: Trevor Kilpatrick, Parkville (AU); Michele Denise Binder, Skye (AU); Zhi-Ming Gerry Ma, Bulleen (AU)

(73) Assignee: HOWARD FLOREY INSTITUTE, Parkville, Victoria (AU)

(21) Appl. No.: 14/110,685

(22) PCT Filed: Apr. 4, 2012
(86) PCT No.: PCT/AU12/00348

§ 371 Date: Mar. 14, 2014

Related U.S. Application Data
(60) Provisional application No. 61/473,298, filed on Apr. 8, 2011.

(51) Int. Cl. G01N 33/566

(52) U.S. Cl. CPC G01N 33/566
USPC 424/85.6; 435/7.92; 436/501

(57) ABSTRACT

The present disclosure relates to the use of TAM receptor ligands, metabolites, precursor and binding partners thereof in the field of inflammatory neuropathology. This includes the early diagnosis and monitoring of an inflammatory neuropathology as well as screening for medicaments used in the treatment and prophylaxis of such a condition. The method comprises screening blood fluid from a subject for a TAM receptor ligand, or metabolite, or precursor thereof wherein an altered level of the ligand or its metabolite or precursor relative to a control is indicative of the presence of an inflammatory neuropathological disease or condition or a likelihood of developing the same. Diagnostic kits, high throughput screening assays and therapeutic compositions for inflammatory neuropathies are also taught herein.

This application is associated with and claims priority from U.S. Provisional Patent Application No. 61/473,298, filed on 8 Apr. 2011, entitled “A method of diagnosis and treatment”, the entire contents of which, are incorporated herein by reference.

### FIELD

The present disclosure relates generally to the field of inflammatory neuropathology including the early diagnosis and monitoring of an inflammatory neuropathology as well as screening for medicaments useful in the treatment and prophylaxis of such a condition. Diagnostic kits, high through-put screening assays, and therapeutic compositions for inflammatory neuropathies are also taught herein.

### BACKGROUND

Bibliographic details of the publications referred to by author in this specification are collected alphabetically at the end of the description.

Reference to any prior art in this specification is not, and should not be taken as an acknowledgment or any form of suggestion that this prior art forms part of the common general knowledge in any country.

Receptor protein tyrosine kinases (PTKs) are cell surface transmembrane receptors which, upon binding of an extracellular ligand, triggers receptor dimerization and kinase activity. Signal transduction cascades initiated by PTK activation control a range of cellular processes including cell differentiation and apoptosis. One particular group of PTK receptors is the TAM family of receptors (Lai and Lemke (1991) Neuron 6:691-704). Three members of this family have been identified to date designated Axl, Mertk and Tyro3 (reviewed by Lemke and Rothlin (2008) Nature Reviews (Immunology) 8:327-336). TAM receptor-mediated signaling is associated with tissue homeostasis in the nervous, reproductive and vascular systems. TAM signaling is also important in regulating innate immune systems including inhibiting the inflammatory response to pathogens and apoptotic cells by dendritic cells and macrophages and maturation of natural killer cells.

TAM receptors and their ligands crystallise as homo- and hetero-dimers. Each monomeric form of the receptor comprises an N-terminal region, a Gla-domain, a EGF-like domain, an immunoglobulin-like domain, a transmembrane domain and a PTK domain (Lemke and Rothlin, (2008) supra). Two TAM receptor ligands have been identified to date, Protein S and growth arrest-specific 6 (GAS6).

Demyelinating disease is a nervous system disorder in which the myelin sheath of neurons is damaged. This reduces signal transmission in affected nerves causing inter alia impairment of sensation, movement and cognition. Demyelinating disease encompasses multiple sclerosis and other idiopathic inflammatory demyelinating diseases. Oligodendrocytes are a major cell type damaged in these types of disease conditions. Hence, the term “oligodendrocyte disease” is used to define demyelinating diseases which affect the oligodendrocytes and their ability to interact with various cell types in the demyelinating area. The TAM family of receptors has been found to be expressed in the nervous system including oligodendrocytes (Binder et al. (2008) The Journal of Neuroscience 28(2):5195-5206). Oligodendrocyte death is an early event in demyelinating disease (Barnett and Prineas (2004) Ann. Neurol 55:459-468).

### SUMMARY

A method for detecting the presence of an inflammatory neuropathological disease or condition in a subject is provided comprising screening blood plasma or other suitable blood fluid from the subject for a TAM receptor ligand wherein an altered level of the ligand relative to a control is indicative of the presence of an inflammatory neuropathological disease or condition or a likelihood of developing same or defines a particular subset of patients with a demyelination event. The TAM receptor ligand may be a free ligand or a complexed ligand or both. In an embodiment, the TAM receptor ligand is Protein S or GAS6 or a metabolite or precursor thereof. Without intending to limit the present embodiments to any one theory or mode of action, the presence of altered levels of a TAM receptor ligand in blood plasma relative to a healthy control is proposed to be indicative of a response to injury, trauma or neurodegeneration. Altered levels of expression of a TAM receptor in blood cells is also indicative of a disease condition or a particular subset of patients.

In an embodiment, the TAM receptor ligand is free Protein S. In another embodiment, the TAM receptor ligand is GAS6. The TAM receptor ligand may be elevated or reduced, depending on the condition or subset of patients.

The subject may be a human or a non-human primate or other suitable animal model.

Reference to an inflammatory neuropathological disease or condition includes multiple sclerosis (MS), oligodendrocyte disease, acute disseminated encephalomyelitis, optic neuropathy (including neuromyelitis optic with transient autonomic disturbances). Devic's neuromyelitis optica, tropical spastic paraparesis, non-compressive myelopathies. concentric sclerosis, diffuse sclerosis acute hemorrhagic leukoencephalopathy, metachromatic leukodystrophy, leucoareosis, acute discriminated encephalomyelitis, progressive multifocal leukoencephalopathy, multisystem entrophy, as well as any form of brain trauma resulting in white matter such as stroke or physical injury.

In an embodiment, the inflammatory neuropathological condition or disease is selected from MS, oligodendrocyte disease, stroke or brain trauma.

Conveniently, the TAM receptor ligand or its metabolite or precursor is identified by an immunoassay such as but not limited to sandwich ELISA. In relation to the latter, the blood plasma or other suitable blood fluid is contacted with an immobilized antibody or antigen-binding fragment thereof specific for the TAM receptor ligand or its metabolite or precursor for a time and under conditions sufficient for the antibody to capture the ligand and then the captured ligand identified by contacting the captured ligand with an antibody or antigen-binding fragment labeled with a reporter molecule or component of a signal generating pathway and then the reporter molecule or generated signal is detected. A useful labeled antibody is an antibody or antigen-binding fragment labeled with horseradish peroxidase which, in the presence of a chromogenic substrate and hydrogen peroxide generates a colored reaction product.

Another aspect enabled herein is the use of a binding partner of a TAM receptor ligand, such as Protein S or GAS6, or a metabolite or precursor thereof in the manufacture of a diagnostic assay to detect levels of the ligand in blood plasma which is indicative of the presence or absence of an inflammatory neuropathology in a subject. In an embodiment, the binding partner is an antibody specific to the TAM receptor ligand or an antigen-binding fragment of the antibody. In an embodiment, the proteins or GAS6 or both is/are elevated compares to a normal control. A normal control is a statistically validated level in a healthy population. In another embodiment, Protein S or GAS6 or both is/are reduced relative to the normal control. Hence, the assay enabled herein includes the stratification of subjects into the presence, severity, stage or subset of a demyelinating condition.

The ability to detect early stage demyelination enables therapy to be initiated expeditiously. This would include the administration of interferon β o r other immunomodulatory agents known to influence the clinical course of inflammatory neuropathological disease including demyelinating disease.

Another aspect taught herein provides a method of treating, controlling or preventing development of an inflammatory neuropathological disease or condition, the method comprising measuring the level of a TAM receptor ligand in a subject and comparing the level to a control, the control being a level from a normal, healthy subject or population of subjects or a level from the same subject as being treated taken at an earlier time point, and if the level of the TAM receptor ligand is elevated compared to the control administering to the subject an effective amount of an agent selected from:

(i) interferon β or another immunomodulatory agent which influences the clinical course of the inflammatory neuropathological disease or condition;

(ii) an agent which reduces demyelination leading to a reduction in plasma levels of the TAM receptor ligand; and

(iii) an agent which normalizes levels of the TAM receptor ligand whilst not inducing or exacerbating inflammatory neuropathology.

In an embodiment, “normalizes” means elevating or reducing levels of the TAM receptor ligand to the levels of a normal, healthy control. Further taught herein is small molecule screening or small molecule design which effect modulation of TAM receptor-mediated signaling by interacting with the TAM receptor, its ligand or competes, for binding between the TAM receptor and its ligand.

Abbreviations used herein are summarized in Table 1.

TABLE 1
Abbreviations

| Abbreviation | Definition |
|---|---|
| Axl | A TAM receptor |
| EAE | Experimental autoimmune encephalitis |
| GAS6 | Ligand of a TAM receptor (Growth arrest gene 6) |
| Mertk | A TAM receptor |
| MS | Multiple sclerosis |
| PEG | Polyethylene glycol |
| Protein S | Ligand of a TAM receptor |
| PTK | Protein tyrosine kinase |
| Tyro3 | A TAM receptor |

### BRIEF DESCRIPTION OF THE FIGURES

FIG. 1A is a graphical representation of levels of blood plasma total Protein S (ProS) in control and multiple sclerosis (MS) patients represented as a percentage or normal Protein S levels.

FIG. 1B is a graphical representation of levels of blood plasma free Protein S (ProS) in control and multiple sclerosis (MS) patients represented as a percentage of normal Protein S levels. Blood plasma was first subjected to polyethylene glycol (PEG) treatment to precipitate Protein S complexed to C4b.

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method step or group of elements or integers or method steps.

As used in the subject specification, the singular forms “a”, “an” and “the” include plural aspects unless the context clearly dictates otherwise. Thus, for example, reference to “a ligand” includes a single ligand, as well as two or more ligands; reference to “an inflammatory neuropathological disease or condition” includes a single inflammatory neuropathological disease or condition, as well as two or more inflammatory neuropathological diseases or conditions; reference to “the embodiment” includes single or multiple aspects of an embodiment taught herein; and so forth.

It is determined herein that blood plasma levels of a TAM ligand are altered during or prior to development of an inflammatory neuropathological disease or condition such as during or prior to oligodendrocyte disease including inflammatory demyelinating disease. The altered levels may also define a subset of patients. By “altered” means elevated or reduced. “Elevated” means that there is enhanced or upregulated levels relative to levels observed in subjects with no evidence of an inflammatory neuropathological disease or condition or relative to the levels of concentrations obtained from that particular subject's plasma in the past (i.e. this is regarded as the “control”). “Reduced” means there is a diminished or down-regulated level relative to the normal healthy control. Without intending to limit the present embodiments to any one theory or mode of action, levels of a TAM receptor ligand are elevated in blood plasma in subjects with an inflammatory neuropathological disease or condition or who are at risk of developing same. Levels can be reduced in a subset of such patients. TAM receptor ligands contemplated herein are Protein S and GAS6. Also contemplated herein include metabolites and precursor forms of Protein S and GAS6 as well as recombinant forms for use in an assay. For convenience, reference to “Protein S” and “GAS6” and “ligand” include metabolites and precursor and recombinant forms thereof that may also be in blood plasma or other suitable blood fluid or used in an assay.

Accordingly, a method is taught for detecting the presence of an inflammatory neuropathological disease or condition in a subject, the method comprising screening blood plasma or other suitable blood fluid from the subject for a TAM receptor ligand or a metabolite or precursor thereof wherein an altered level of the ligand relative to a control is indicative of the presence of the inflammatory neuropathological disease or condition or a likelihood of developing same.

Enabled herein is a method for detecting the presence of an inflammatory neuropathological disease or condition in a subject, the method comprising screening blood plasma or other suitable blood fluid from the subject for a TAM receptor ligand or a metabolite or precursor thereof wherein an elevated level of the ligand relative to a control is indicative of the presence of the inflammatory neuropathological disease or condition or a likelihood of developing same.

Enabled herein is a method for detecting the presence of an inflammatory neuropathological disease or condition in a subject, the method comprising screening blood plasma or other suitable blood fluid from the subject for a TAM receptor ligand or a metabolite or precursor thereof wherein a reduced level of the ligand relative to a control is indicative of the presence of the inflammatory neuropathological disease or condition or a likelihood of developing same.

Also taught herein is a method for detecting the presence of an inflammatory neuropathological disease or condition in a subject, the method comprising screening blood plasma or other suitable body fluid from the subject for a TAM receptor ligand selected from Protein S and GAS6 or a metabolite or precursor thereof wherein an altered level of the ligand relative to a control is indicative of the presence of the inflammatory neuropathological disease or condition or a likelihood of developing same.

Also taught herein is a method for detecting the presence of an inflammatory neuropathological disease or condition in a subject, the method comprising screening blood plasma or other suitable body fluid from the subject for a TAM receptor ligand selected from Protein S and GAS6 or a metabolite or precursor thereof wherein an elevated level of the ligand relative to a control is indicative of the presence of the inflammatory neuropathological disease or condition or a likelihood of developing same.

Also taught herein is a method for detecting the presence of an inflammatory neuropathological disease or condition in a subject, the method comprising screening blood plasma or other suitable body fluid from the subject for a TAM receptor ligand selected from Protein S and GAS6 or a metabolite or precursor thereof wherein an a reduced level of the ligand relative to a control is indicative of the presence of the inflammatory neuropathological disease or condition or a likelihood of developing same.

Further enabled herein, is a method for detecting the presence of an inflammatory neuropathological disease or condition in a subject, the method comprising screening blood plasma or other suitable body fluid from the subject for Protein S or a metabolite or precursor thereof wherein an altered level of Protein S relative to a control is indicative of the presence of the inflammatory neuropathological disease or condition or a likelihood of developing same. In an embodiment, the level of Protein S is elevated. In another embodiment, it is reduced.

The TAM receptor ligand may be in a free form or complexed to another molecule. For example, Protein S exists in a free form or complexed to C4b. Hence, total Protein S (free+complexed), free Protein S and/or complexed Protein S may be measured.

A “control” is the statistically validated level of the TAM receptor ligand in a healthy subject or population of subjects with no evidence of an inflammatory neuropathological disease or condition. A “control” is also the level of TAM receptor ligand in blood plasma measured previously in blood plasma from the same subject. Hence, an elevation of time compared to the level previously measured would be an indication of the development or progression of an inflammatory neuropathological disease or condition. Hence, a “control” may be from a normal, healthy subject or population of subjects or an earlier level from the same subject. Conveniently, the level of TAM receptor ligand in blood plasma or other suitable blood fluid is expressed as a percentage of the levels in a control blood plasma sample. A subject is deemed to be at risk of having or developing an inflammatory neuropathology when the levels of the TAM receptor ligand or its metabolite or precursor reached >105% of normal levels. By “>105%” includes from about 105% to 500% including 105%, 110%, 120%, 150%, 200%, 300%, 400% and 500% of normal levels. A subset of patients has a reduced level of TAM receptor ligand. By “reduced” is meant from about 20% to 95% of normal levels.

Hence, a method is provided for detecting the presence of an inflammatory neuropathological disease or condition in a subject, the method comprising screening blood plasma or other suitable blood fluid from the subject for a TAM receptor ligand or metabolite or precursor thereof wherein a level of the ligand greater than 105% of the level in a control is indicative of the presence of the inflammatory neuropathological disease or condition or a likelihood of developing same.

Another aspect taught herein is a method is provided for detecting the presence of an inflammatory neuropathological disease or condition in a subject, the method comprising screening blood plasma or other suitable blood fluid from the subject for a TAM receptor ligand selected from Protein S and GAS6 or metabolite or precursor thereof wherein a level of the ligand greater than 105% of the level in a control is indicative of the presence of the inflammatory neuropathological disease or condition or a likelihood of developing same.

Also enabled herein is a method is provided for detecting the presence of an inflammatory neuropathological disease or condition in a subject, the method comprising 30. screening blood plasma or other suitable blood fluid from the subject for Protein S or metabolite or precursor thereof wherein a level of the Protein S greater than 105% of the level in a control is indicative of the presence of the inflammatory neuropathological disease or condition or a likelihood of developing same.

A method is also provided for detecting the presence of an inflammatory neuropathological disease or condition in a subject, the method comprising screening blood plasma or other suitable blood fluid from the subject for a TAM receptor ligand or metabolite or precursor thereof wherein a level of the ligand from 20-95% of the level in a control is indicative of the presence of the inflammatory neuropathological disease or condition or a likelihood of developing same.

Another aspect taught herein is a method for detecting the presence of an inflammatory neuropathological disease or condition in a subject, the method comprising screening blood plasma or other suitable blood fluid from the subject for a TAM receptor ligand selected from Protein S and GAS6 or metabolite or precursor thereof wherein a level of the ligand from 20-95% of the level in a control is indicative of the presence of the inflammatory neuropathological disease or condition or a likelihood of developing same.

Also enabled herein is a method for detecting the presence of an inflammatory neuropathological disease or condition in a subject, the method comprising screening blood plasma or other suitable blood fluid from the subject for Protein S or metabolite or precursor thereof wherein a level of the Protein S from 20-95% of the level in a control is indicative of the presence of the inflammatory neuropathological disease or condition or a likelihood of developing same.

As indicated above, “greater than 105%” includes from 105% to 500%. such as 105, 106, 107, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160. 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205. 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500%.

From “20-95” means 20, 21, 22, 23, 24, 25, 26, 27, 28, 29, 30, 31, 32, 33, 34, 35. 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80. 81, 82, 83, 84, 85, 86, 87, 88, 89, 90, 91, 92, 93, 94 or 95%.

Alternatively, the level of expression of a TAM receptor such as Axl, Mertk and Tyro3 is measured. Altered levels of these receptors can define a subset of patients with a demyelination condition.

Disease and conditions contemplated herein included multiple sclerosis (MS), oligodendrocyte disease, acute disseminated encephalomyelitis, optic neuropathy (including neuromyelitis optic with transient autonomic disturbances), Devic's neuromyelitis optica, tropical spastic paraparesis, non-compressive myelopathies, concentric sclerosis, diffuse sclerosis acute hemorrhagic leukoencephalopathy, metachromatic leukodystrophy, leucoareosis, acute discriminated encephalomyelitis, progressive multifocal leukoencephalopathy, multisystem entrophy, as well as any form of brain trauma resulting in white matter such as stroke or physical injury. All these disease conditions are encompassed by the terms “inflammatory neuropathological disease or condition”, “inflammatory neuropathology” and “neurodegenerative disease or condition”.

Oligodendrocyte diseases include a disease which results in apoptosis or death of oligodendrocytes or other cytotoxic events involving oligodendrocytes as well as oligodendrocyte cell cycle arrest or demyelination. The inflammatory neuropathology may also involve microglial activation.

The term “oligodendrocyte” or its plural form “oligodendrocytes” means those neural cells which provide support for axons and which produce the myelin sheath. Oligodendrocytes form segments of myelin sheaths of numerous neurons. Oligodendrocytes are a class of glial cells.

An example of a demyelinating disease is an inflammatory idiopathic demyelinating disease such as MS.

Therefore, enabled herein is a method for detecting the presence of MS in a subject.

the method comprising screening blood plasma or other suitable body fluid from the subject for a TAM receptor ligand or a metabolite or precursor thereof wherein elevated levels of the ligand is indicative of the presence of MS or a risk of developing same.

As indicated above, examples of TAM receptor ligands include Protein S and GAS6 or free and/or complexed forms thereof. A subset of MS patients may also be defined where there is a reduced level of Protein S or GAS6.

The methodology taught herein is also applicable to screening subjects for an inflammatory neuropathological disease or condition. Hence, this aspect contemplates a method for screening a subject for an inflammatory neuropathological disease or condition, the method comprising determining the level of a TAM receptor ligand or a metabolite or precursor thereof in blood plasma or other suitable blood fluid wherein an altered level of the ligand relative to a control is indicative that the subject has or may develop an inflammatory neuropathological disease or condition.

This aspect includes detecting Protein S and/or GAS6 or their metabolites or precursors. By “altered” means elevated or reduced.

The subject may also be exposed to other diagnostic tests including behavioral tests, cognitive tests, or assays of other biomarkers.

A “TAM receptor” includes monomeric or multimeric (homo- or hetero-meric) forms of Mertk, Axl and Tyro3. A TAM receptor ligand includes Protein S and GAS6. Levels of expression of Axl, Mertk and/or Tyro3 may also be measured or measured in place of the ligand.

The method taught herein may be referred to inter alia as an assay, screen, method, test, system, diagnosis, prognosis, bioassay, determination or report. The method is useful for diagnosing an inflammatory neuropathological disease or condition as well as monitoring disease progression such as following medicament intervention or behavioral modification. The assay can also be used to screen for medicaments which lower TAM receptor ligand levels in blood plasma which is indicative of amelioration of the demyelinating events.

A method is taught herein for monitoring progression of an inflammatory neuropathological disease or condition in a subject, the method comprising screening blood plasma or other suitable blood fluid in the subject for a TAM receptor ligand or a metabolite or precursor thereof over time wherein a change in the level of ligand within a time period is indicative of disease progression. In an example, an increasing level of the ligand over time is indicative of the disease progressing. Alternatively, a decreasing or normalization of the level is indicative of the amelioration of the disease or condition.

Yet another aspect enabled herein is a method for detecting the presence of an inflammatory neuropathological condition or disease or monitoring its progression in a subject, the method comprising screening blood plasma or other suitable blood fluid from the subject for the level of a TAM receptor ligand wherein an altered level of the ligand compared to a control is indicative of the presence of an inflammatory neuropathological disease or condition or a likelihood of developing same or the continued progression of the disease or condition.

Yet another aspect enabled herein is a method for detecting the presence of an inflammatory neuropathological condition or disease or monitoring its progression in a subject, the method comprising screening blood plasma or other suitable blood fluid from the subject for the level of a TAM receptor ligand wherein an elevated level of the ligand compared to a control is indicative of the presence of an inflammatory neuropathological disease or condition or a likelihood of developing same or the continued progression or the disease or condition.

Yet another aspect enabled herein is a method for detecting the presence of an inflammatory neuropathological condition or disease or monitoring its progression in a subject, the method comprising screening blood plasma or other suitable blood fluid from the subject for the level of a TAM receptor ligand wherein an reduced level of the ligand compared to a control is indicative of the presence of an inflammatory neuropathological disease or condition or a likelihood of developing same or the continued progression of the disease or condition.

Screening for elevated levels of a TAM, receptor ligand may be by any means such as determining protein levels or mRNA levels or other genetic indicators of apoptosis or cell death. Also taught herein is the determination of minimal residual disease (MRD) in inflammatory neuropathological conditions. This applies to the minimal detectable level of demyelination events. This enables clinicians to monitor biochemically a subject which has improved or deteriorated neurological function after treatment.

Conveniently, the assay is conducted using blood plasma. However, the assay described herein contemplates any fluid in which the TAM receptor ligand may be present. Information provided by the assay enables the clinician, alone or in combination with other symptoms of an inflammatory neuropathological condition, to make a determination of the, level of severity of the disease or condition and its progression.

The level of TAM receptor ligand provides an indication of level of stress in the nervous system. The assay herein enables early detection of an inflammatory neuropathology or a likelihood for its development. The ligand, such as Protein S or GAS6, may be screened or a metabolite or precursor form thereof may be assayed.

The present specification teaches the use of a binding, partner of a TAM receptor ligand or mRNA in the manufacture of an assay to detect the level of the ligand in blood plasma which is indicative of the presence or absence of an inflammatory neuropathology in a subject. Conveniently, the binding partner to a ligand protein is an antibody or an antigen-binding fragment thereof. The inflammatory neuropathology includes an inflammatory idiopathic demyelination disease such as MS or other disorders involving oligodendrocytes.

The TAM receptor ligand may be detected by any number of means. One convenient assay involves an antibody or antigen-binding portion thereof specific for the TAM receptor ligand immobilized to a solid support in a reaction vessel. The solid support includes the wall of the vessel.

In an embodiment, a whole blood sample collected from the subject is deposited into a blood collection tube. A blood collection tube includes a blood draw tube or other similar vessel. Conveniently, when the sample is whole blood, the blood collection tube is heparinized. Alternatively, heparin is added to the tube after the blood is collected. The blood collection tube or other tube to which the whole blood is transferred is then subjected to centrifugal action to pellet the cells and the plasma is then removed as supernatant fluid.

The use of blood collection tubes is compatible with standard automated laboratory systems and these are amenable to analysis in large-scale and random access sampling. Blood collection tubes also minimize handling costs and reduce laboratory exposure to whole blood and plasma and, hence, reduce the risk of laboratory personnel from contracting a pathogenic agent such as Human Immunodeficiency virus (HIV) or Hepatitis B virus (HBV) or Hepatitis C virus (HCV).

Combining the incubation step with the collection tube is a feature which can add to the convenience of the assay.

The plasma and the immobilized antibody or antigen-binding fragment thereof are incubated to enable binding between free or complexed ligand and the antibody.

The incubation step may be from 1 to 50 hours, such as I to 40 hours or 8 to 24 hours or a time period in between including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours. A period of 24 hours is particularly convenient.

If free ligand only is to be assayed, then the plasma may first be subjected to a polyethylene glycol (PEG) precipitation step to remove any protein complex comprising TAM receptor ligand.

After the incubation step, the plasma fluid is removed and the presence of any captured ligand determined.

The detection of captured TAM receptor ligand may be measured by any number of means. Techniques contemplated herein include, for example, ELISA, sandwich assays, radioimmunoassay and chemiluminesence immunoassay. Reference to “antibodies” includes parts of antibodies, recombinant or synthetic antibodies and hybrid and single chain antibodies.

Both polyclonal and monoclonal antibodies specific for the TAM receptor ligand or its metabolite or precursor are obtainable by immunization with the TAM receptor ligand or metabolite or precursor form or an antigenic fragment thereof and either type is utilizable for immunoassays. Methods of obtaining both types of sera are well known in the art. Polyclonal sera are less preferred but are relatively easily prepared by injection of a suitable laboratory animal with an effective amount of the ligand or metabolite or precursor form, or antigenic part thereof, collecting serum from the animal and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is useful because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect enabled herein, therefore, is a method for detecting a captured TAM receptor ligand or metabolite or precursor thereof in a plasma sample from a subject, the method comprising contacting the sample or an aliquot of the sample with an antibody specific for the ligand or its metabolite or precursor or an antigenic fragment thereof for a time and under conditions sufficient for an antibody-ligand complex to form, and then detecting the complex. This is in effect an immunosandwich ELISA with an antibody specific for the ligand binding to the ligand already captured by an immobilized antibody.

Plasma volumes may be from about 0.5 μl to 200 ml. Examples include 0.5 μl, 1.5 μl, 10 μl, 20 μl, 50 μl, 100 μl, 500 μl, 1 ml, 5ml, 10 ml, and 20 ml. The present disclosure also enables the use of acoustic microstreaming to improve the mixing of components in the assay. Acoustic microstreaming is disclosed in International Patent Application No. PCT/AU01/00420 and in Petkovic-Duran et al. (2009) Biotechniques 47:827-834.

In terms of acoustic microstreaming, contemplated herein is a method of assaying blood plasma or other blood fluid for a TAM receptor ligand or metabolite or precursor thereof in a vessel, the method comprising providing from about 0.5 μl to 150 μl of plasma fluid in the vessel having antibody or antigen-binding fragments thereof immobilized to a solid support in the vessel so as to establish a discontinuity in acoustic impedance and applying an acoustic signal to cause mixing within the fluid. A second acoustic signal may also be applied, the first and second signals having respective frequencies each selected from about 1 Hz to about 20,000 Hz in an alternating manner to effect chaotic mixing within the fluid. This enables adequate mixing for the ligand or its metabolite or precursor form to come into contact with and bind to the immobilized antibody. Captured ligand is then detected as above.

A “sample” includes blood plasma sample or other suitable fluid comprising the TAM receptor ligand or a metabolite or precursor form thereof. This method includes micro-arrays, macro-arrays and nano-arrays on planar or spherical solid supports. A micro- or macro-array is useful. A “sample” also includes a small volume sample of from about 0.5 μl to 1000 μl including 5 μl, 10 μl, 20 μl, 50 μl and 100 μl as well as larger volumes such as from 1 ml to about 200 ml such as 1 ml, 2 ml, 5 ml, 10 ml or 20 ml.

A wide range of immunoassay techniques are available as can be seen by reference to U.S. Pat. Nos. 4,016,043, 4,424,279 and 4,018,653.

The following is a description of one type of assay. An unlabeled antibody is immobilized on a solid substrate and the sample to be tested for the TAM receptor ligand or a metabolite or precursor form thereof brought into contact with the bound molecule. After a suitable period of incubation, for a period of time sufficient to allow formation of an antibody-ligand complex, a second antibody specific to the ligand, labeled with a reporter molecule capable of producing a detectable signal or a component in a signal generating pathway or reaction, is then added and incubated, allowing time sufficient for the formation of another complex of antibody-ligand-labeled antibody. Any unreacted material is washed away, and the presence of the captured ligand is determined by observation of a signal produced by the reporter molecule or by the signal generated in the signal generating pathway. The results may either be qualitative, by simple observation of the visible signal, or may be quantitated by comparing with a control sample containing known amounts of ligand. This generalized technique is well known to those skilled in the art as would be any of a number of variations.

In these assays, a first antibody having specificity for the instant ligand is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, spheres, discs of microplates, or any other surface suitable for conducting an immunoassay. Conveniently, the solid phase is the internal surface of the vessel. The binding processes are well known in the art and generally consist of cross-linking covalently binding or physically adsorbing, the polymer-antibody complex is washed in preparation for the test sample. An aliquot of the plasma sample to be tested is then added to the solid phase complex and incubated for a period of time sufficient (e.g. 1-50 hours or where more convenient, overnight) and under suitable conditions (e.g. for about 20° C. to about 40° C.) to allow binding of ligand to be immobilized antibody. Following the incubation period, the vessel is washed and incubated with a second antibody specific for a portion of the ligand. The second antibody is linked to a receptor molecule or a component in a signal generating pathway or reaction which is used to indicate the binding of the second antibody to the ligand.

There are many variations to this assay. One variation is a simultaneous assay where all or many of the components are substantially simultaneously admixed. Furthermore, binding of an antibody to a ligand may be determined by binding of a labeled antibody directed to the first mentioned antibody.

By “reporter molecule” and “component in a signal generating pathway or reaction” as used are meant a molecule which, by its' chemical nature, provides either directly or via other components an analytically identifiable signal which allows the detection of a ligand-bound antibody complex. Detection may be either qualitative or quantitative. The most commonly used reporter molecules in this type of assay are either enzymes, fluorophores or radionuclide containing molecules (i.e. radioisotopes) and chemiluminescent molecules. Examples of suitable fluorophores are provided in Table 2. In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include hydrogen peroxidase and alkaline phosphatase. It is also possible to employ fluorogenic substrates, which yield a fluorescent product rather than the chromogenic substrates noted above. In all cases, the enzyme-labeled antibody is added to the first antibody-antigen complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample. Again, the present disclosure extends to a substantially simultaneous assay.

Alternately, fluorescent compounds, such as fluorescein and rhodamine, may he chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. The fluorescent labeled antibody is allowed to bind to the first antibody-antigen complex. After washing off the unbound reagent, the remaining tertiary complex is then exposed to the light of the appropriate wavelength the fluorescence observed indicates the presence of the antigen of interest. Immunofluorescene and enzyme immunoassay techniques are both very well established in the art and are particularly preferred for the present method. However, other reporter molecules, such as radioisotope, chemiluminescent or bioluminescent molecules, may also be employed.

There are a range of other detection systems which may be employed including colloidal gold and all such detection systems are encompassed by the present disclosure.

The present disclosure also contemplates genetic assays such as involving PCR analysis to detect genetic material encoding a TAM receptor ligand.

In one embodiment, PCR is conducted using pairs of primers, one or both of which are generally labeled with the same or a different reporter molecule capable of giving a distinguishable signal. Examples of suitable fluorophores may be selected from the list given in Table 2. Other labels include luminescence and phosphorescence as well as infrared dyes. These dyes or fluorophores may also be used as reporter molecules for antibodies.

TABLE 2
List of suitable fluorophores

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

Any suitable method of analyzing fluorescence emission is encompassed herein. In this regard, techniques taught herein include but are not restricted to 2-photon and 3-photon time resolved fluorescence spectroscopy as, for example, disclosed by Lakowicz et al. (1997) Biophys. J. 72:567, fluorescence lifetime imaging as, for example, disclosed by Eriksson et al. (1993) Biophys. J. 2:64 and fluorescence resonance energy transfer as, for example, disclosed by Youvan et al. (1997) Biotechnology et elia 3:1-18.

Luminescence and phosphorescence may result respectively from a suitable luminescent or phosphorescent label as is known in the art. Any optical means of identifying such label may be used in this regard.

Infrared radiation may result from a suitable infrared dye. Exemplary infrared dyes that may be employed in the present disclosure include but are not limited to those disclosed in Lewis et al. (1999) Dyes Pigm. 42(2):197, Tawa et al. Mater. Res. Soc. Syrup. Proc. 488 [Electrical, Optical and Magnetic Properties of Organic Solid-State Materials IV], 885-890, Daneshvar et al. (1999) J. Immunol. Methods 226(1-2):119-128, Rapaport et al. (1999) Appl. Phys. Lett. 74(3):329-331 and Durig et al. (1993) J. Raman Spectrosc. 24(5):281-285. Any suitable infrared spectroscopic method may be employed to interrogate the infrared dye. For instance, fourier transform infrared spectroscopy as, for example, described by Rahman et al. (1998) J. Org. Chem. 63:6196 may be used in this regard.

Suitably, electromagnetic scattering may result from diffraction, reflection, polarization or refraction of the incident electromagnetic radiation including light and X-rays. Such scattering can be used to quantitate the level of level of protein or mRNA.

Flow cytometry is particularly useful in analyzing fluorophore emission.

As is known in the art, flow cytometry is a high throughput technique which involves rapidly analyzing the physical and chemical characteristics of particles (e.g. labeled proteins) as they pass through the path of one or more laser beams while suspended in a fluid stream. As each particle intercepts the laser beam, the scattered light and fluorescent light emitted by each cell or particle is detected and recorded using any suitable tracking algorithm as, for example, described hereunder.

A modern flow cytometer is able to perform these tasks up to 100,000 cells/particles s⁻¹. Through the use of an optical array of filters and dichroic mirrors, different wavelengths of fluorescent light can be separated and simultaneously detected. In addition, a number of lasers with different excitation wavelengths may be used. Hence, a variety of fluorophores can be used to target and examine, for example, different ligands within a sample or ligands from multiple subjects.

Suitable flow cytometers which may be used in the methods of the present disclosure include those which measure five to nine optical parameters (see Table 3) using a single excitation laser, commonly an argon ion air-cooled laser operating at 15 mW on its 488 nm spectral line. More advanced flow cytometers are capable of using multiple excitation lasers such as a HeNe laser (633 nm) or a HeCd laser (325 nm) in addition to the argon ion laser (488 or 514 nm).

TABLE 3
Exemplary optical parameters
which may be measured by a flow cytometer.

| | | Detection angle form | Wavelength |
|---|---|---|---|
| Parameter | Acronym | incident laser beam | (nm) |
| Forward scattered light | FS | 2-5° | 488* |
| Side scattered light | SS | 90° | 488* |
| “Green” fluorescence | FL1 | 90° | 510-540^{†} |
| “Yellow” fluorescence | FL2 | 90° | 560-580^{†} |
| “Red” fluorescence | FL3 | 90° | >650^{#} |

*using a 488 nm excitation laser
^{†}width of bandpass filter
^{#}longpass filter

For example, Biggs et al. (1999) Cytometry 36:36-45 have constructed an 11-parameter flow cytometer using three excitation lasers and have demonstrated the use of nine distinguishable fluorophores in addition to forward and side scatter measurements for purposes of immunophenotyping (i.e. classifying) particles. Selection of parameters can be adequately used depends heavily on the extinction coefficients, quantum yields and amount of spectral overlap between all fluorophores (Malemed et al. (1990) “Flow cytometry and sorting”. 2^{nd} Ed., New York, Wiley-Liss). It will be understood that the present disclosure is not restricted to any particular flow cytometer or any particular set of parameters. In this regard, the disclosure also contemplates use in place of a conventional flow cytometer, a microfabricated flow cytometer as, for example, disclosed by Fu et al. (1999) Nature Biotechnology 17:1109-1111.

The assay enabled herein may be automated or semi-automated for high throughput screening of TAM receptor ligands from a number of subjects. The automation is conveniently controlled by computer software.

The present disclosure further contemplates web-based and non-web-based systems where data on the levels of TAM receptor ligands of a subject are provided by a client server or other architecture platform to a central processor which analyses and compares to a control and optionally considers other information such as patient age, sex, weight and other medical conditions and then provides a report, such as, for example, a risk factor for disease severity or progression or status or an index of probability of disease development. A business method is, therefore, also provided whereby blood is collected in transportable tubes which is then analyzed for levels of TAM receptor ligands and the results then sent in the form of an electronic report via a client server or other architecture platform to a clinical care provider.

Hence, knowledge-based computer software and hardware also form part of the present disclosure. This facilitates clinical care to ascertain the state and progression of a neurodegenerative disease or condition.

In particular, the assays enabled by the instant disclosure may be used in existing or newly developed knowledge-based architecture or platforms associated with pathology services. For example, results from the assays are transmitted via a communications network (e.g. the internet) or telephone connection to a processing system in which an algorithm is stored and used to generate a predicted posterior probability value which translates to the index of inflammatory neuropathy which is then forwarded to an end user in the form of a diagnostic or predictive report. This report may also form the basis of clinical care management and personalized medicine.

The assay may, therefore, be in the form of a kit or computer-based system which comprises the reagents necessary to detect the concentration of the TAM receptor ligand and the receptor hardware and/or software to facilitate determination and transmission of reports to a clinician.

For example, the present disclosure contemplates a method of allowing a user to determine the status of an inflammatory neuropathology in a subject, the method including:

(a) receiving data in the form of levels or concentrations of a TAM receptor ligand which, relative to a control, provide a correlation as the state of an inflammatory neuropathy in a subject, via a communications network;

(b) processing the subject data via univariate or multivariate analysis to provide value of the progression or state of the inflammatory neuropathology;

(c) determining the status of the subject in accordance with the results of the TAM receptor ligand values in comparison with predetermined values; and

(d) transferring an indication of the status of the subject to the user via the communications network.

As indicated above, a “control” may be the levels in a healthy, normal population or the levels in blood plasma from the same subject measured earlier tin time. Reference to the “univariate” or “multivariate” analysis includes an algorithm which performs the univariate or multivariate analysis function. Reference to a “ligand” includes its metabolite and precursor forms.

The determination of the concentrations or levels of the TAM receptor ligand enables establishment of a diagnostic rule based on the concentrations relative to controls. Alternatively, the diagnostic rule is based on the application of a statistical and machine learning algorithm. Such an algorithm uses relationships between ligand levels and disease status observed in training data (with known disease status) to infer relationships which are then used to predict the status of subjects with unknown status. An algorithm can be employed which provides an index of probability that a subject has a certain level or state of an inflammatory neuropathology. The algorithm performs a univariate or multivariate analysis function.

The levels or concentrations of the TAM receptor ligand provide the input test data referred to herein as a “second knowledge base of data”. The second knowledge base of data either is considered relative to a control or is fed into an algorithm generated by a “first knowledge base of data” which comprise information of the levels of ligand in a subject with a known neurological status. The second knowledge base of data is from a subject of unknown status with respect to neurological status. The output of the algorithm or the comparison to a control is a probability or risk factor, referred to herein as “an index of probability”, of a subject having a certain level or state of inflammatory neuropathy.

In a further embodiment, the present disclosure enables kits for use with the methods described above. In one embodiment, a TAM receptor ligand immunodetection kit is contemplated.

The immunodetection reagents of the kit may take any one of a variety of forms, including those detectable labels that are associated with or linked to the given antibody or antigen, and detectable labels that are associated with or attached to a secondary binding agent. Exemplary secondary agents are those secondary antibodies that have binding affinity for the ligand captured by an immobilized antibody.

Further suitable immunodetection reagents for use in the present kits include the two-component reagent that comprises a secondary antibody that has binding affinity for the captured TAM receptor ligand, along with a third antibody that has binding affinity for the second antibody, the third antibody being linked to a detectable label.

The kits may further comprise a suitably aliquoted composition of TAM receptor ligand, whether labeled or unlabeled, as may be used to prepare a standard curve for a detection assay.

The kits may contain antibody-label conjugates either in fully conjugated form, in the form of intermediates, or as separate moieties to be conjugated by the user of the kit. The components of the kits may be packaged either in aqueous media or in lyophilized form.

The container means of any of the kits generally includes at least one vial, test tube, flask, bottle, syringe or other container means, into which the testing agent, the antibody or antigen may be placed, and generally, suitably aliquoted. Where a second or third binding ligand or additional component is provided, the kit will also generally contain a second, third or other additional container into which this ligand or component may be placed. The kits taught by the present disclosure also typically include a means for containing the antibody, antigen, and any other reagent containers in close confinement for commercial sale. Such containers may include injection or blow-molded plastic containers into which the desired vials are retained.

The assay may also be adapted for screening of agents which ameliorate the inflammatory neuropathological disease or condition which manifests itself in reduced (or reducing) levels of a TAM receptor ligand in blood plasma. The assay may be adapted in any number of ways including large scale screening of blood plasma in test subjects which have been administered a medicament to test for its ability to ameliorate the symptoms of the inflammatory neuropathology. In an example, this may be a clinical trial using volunteer patients with known disease status. The high through put screening system may also be semi-automated to increase the ability to screen large numbers of compounds in large numbers of test subjects. Alternatively, the agent may alter levels of the TAM receptor ligand whilst not inducing an inflammatory response. Examples of agents include small chemical or biological molecules screen for in a library of chemical molecules or designed based on the conformation of the TAM receptor or its ligand.

The subject may be a human or test animal.

Also taught herein is a therapeutic agent identified being useful in the treatment of an inflammatory neuropathological disease or condition as evidenced by its ability to normalize levels of blood plasma TAM receptor ligand. A therapeutic agent is further contemplated which elevates levels of TAM receptor ligand whilst not inducing or exacerbating an adverse inflammatory response.

Hence, a method is provided for the treatment or prophylaxis of an inflammatory neuropathological disease or condition in a subject, the method comprising administering to the subject an effective amount of an agent for a time and under conditions sufficient to ameliorate the inflammatory neuropathological disease or condition, the agent altering blood plasma levels of a TAM receptor ligand. By “reducing blood plasma level” includes reducing levels to control levels, also referred to as normalization of blood plasma levels.

Another aspect provides for the treatment or prophylaxis of an inflammatory neuropathological disease or condition in a subject, the method comprising administering to the subject an effective amount of an agent for a time and under conditions sufficient to ameliorate the inflammatory neuropathological disease or condition, the agent altering levels of a TAM receptor ligand whilst not inducing or exacerbating an inflammatory response. By “altering” includes normalizing to levels in a healthy control.

Hence, a method is provided for the treatment or prophylaxis of an inflammatory neuropathological disease or condition in a subject, the method comprising administering to the subject an effective amount of an agent for a time and under conditions sufficient to ameliorate the inflammatory neuropathological disease or condition, the agent elevating blood plasma levels of a TAM receptor ligand.

Another aspect provides for the treatment or prophylaxis of an inflammatory neuropathological disease or condition in a subject, the method comprising administering to the subject an effective amount of an agent for a time and under conditions sufficient to ameliorate the inflammatory neuropathological disease or condition, the agent reducing levels of a TAM receptor ligand whilst not inducing or exacerbating an inflammatory response.

A method of treating, controlling or preventing development of an inflammatory neuropathological disease or condition, the method comprising measuring the level of a TAM receptor ligand in a subject and comparing the level to a control, the control being a level from a normal, healthy subject or population of subjects or a level from the same subject as being treated taken at an earlier time point, and if the level of the TAM receptor ligand is elevated compared to the control administering to the subject an effective amount of an agent selected from:

(i) interferon β or another immunomodulatory agent which influences the clinical course of the inflammatory neuropathological disease or condition;

(ii) an agent which reduces demyelination leading to a reduction in plasma levels of the TAM receptor ligand; and

(iii) an agent which normalizes levels of the TAM receptor ligand whilst not inducing or exacerbating inflammatory neuropathology.

The terms “compound”, “active agent”, “chemical agent”, “pharmacologically active agent”, “medicament”, “active” and “drug” are used interchangeably herein to refer to such agents which induce a desired pharmacological and/or physiological effect. The desired effect includes reduced release of a TAM receptor ligand. This leads to inducing or promoting oligodendrocyte survival and maintenance, promotion or protection of myelination and/or protection of axons and/or neurons. The terms also encompass pharmaceutically acceptable and pharmacologically active ingredients of those active agents specifically mentioned herein including but not limited to salts, esters, amides, prodrugs, active metabolites, analogs, mimetics functional equivalents and the like. When the terms “compound”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. In an alternative embodiment, the agent elevates levels of TAM receptor ligand without altering inflammation. This is based on the premise that TAM receptor ligands are sentinels of disease. Hence, in an embodiment, the medicament elevates the TAM receptor ligand, such as Protein S or GAS6, without inducing an inflammatory response.

Reference to a “compound”, “active agent”, “chemical agent” “pharmacologically active agent”, “medicament”, “active” and “drug” includes combinations of two or more actives. A “combination” also includes multi-part such as a two-part composition where the agents are provided separately and given or dispensed separately or admixed together prior to dispensation.

The agents, in another embodiment, promote myelination and oligodendrocyte survival.

Reference to promoting oligodendrocyte survival includes reducing oligodendrocyte cytotoxicity or cell cycle arrest as well as promoting oligodendrocyte maintenance; promoting or protecting myelination includes inhibiting, preventing or otherwise reducing demyelination; protection of axons and neurons includes promoting axonal and neuronal repair, function and maintenance. The term “cell cycle arrest” includes cytostasis or other arrest of cell growth (whether cytotoxic or not) and cell senescence.

The terms “effective amount” and “therapeutically effective amount” of an agent as used herein mean a sufficient amount of an agent to provide the desired therapeutic or physiological effect or outcome as indicated above. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or underlying cause, prevention of the occurrence of symptoms of the condition and/or their underlying cause and improvement or remediation or amelioration of damage following a neurodegenerative condition. In general terms, treatment may involve actively reversing a disease or ameliorating symptoms of, for example, oligodendrocyte cell death, senescence or arrest of cell growth, demyelination and/or axonal or neuronal degeneration or modulating an immune response. Amelioration of downstream physiological. psychological or mental conditions is also a useful indicator of treatment. The treatment may result in an immediate effect such as enhancing TAM receptor signaling.

“Treating” a subject, therefore, may involve prevention of a condition or other adverse physiological or psychological event in a susceptible individual as well as treatment of a clinically symptomatic individual by ameliorating the symptoms of the condition.

A “subject” as used herein refers to an animal, preferably a mammal and more preferably human who can benefit from the pharmaceutical agents and formulations and methods of the present disclosure. There is no limitation on the type of animal that could benefit from the presently described pharmaceutical formulations and methods. A subject regardless of whether a human or non-human animal may be referred to as an individual, patient, animal, host or recipient. The compounds and methods of the present disclosure have particular applications in human medicine.

As indicated above, particular animals are humans but other primates such as orangutangs, gorillas and marmosets, macaques, livestock animals, laboratory test animals, companion animals or captive wild animals, as well as avian species may be useful animal models. Whilst humans are the most important subject, non-human animals are useful animal models.

Examples of laboratory test animals include mice, rats, rabbits, guinea pigs, hamsters and non-human primates such as marmosets. Rabbits and rodent animals, such as rats and mice, provide a convenient test system or animal model. Livestock animals include sheep, cows, pigs, goats, horses and donkeys. Non-mammalian animals include avian species, zebrafish, and amphibians including Xenopus spp.

The ability to detect early stage demyelination enables therapy to be initiated expeditiously. This would include the administration of interferon β or other immunomodulatory agent which influences the clinical course of an inflammatory neuropathological disease, such as demyelinating disease.

### EXAMPLES

Aspects taught therein are further described by the following non-limiting Examples.

### Example 1

### Detection of TAM Receptor Ligand

An ELISA based assay is used to detect a TAM receptor ligand. Conveniently, a sandwich ELISA is used. An antibody specific for human Protein S or GAS6 is coated to a solid phase support in a vessel such as a microtiter well. Diluted plasma is added to the vessel and allowed to incubate for a time and under conditions for any Protein S or GAS6 in the plasma to bind to the immobilized antibody, depending on whether the antibody is specific for Protein S or GAS6. The vessel is then emptied and gently washed.

A second anti-Protein S or anti-GAS6 antibody labeled with a reporter molecule or enzyme is then added to the vessel for a time and under conditions sufficient for the labeled antibody to bind to any captured ligand (Protein S or GAS6).

In an example, the second antibody is labeled with horseradish peroxidase. In this example, a chromogenic substrate is added, such as tetramethylbenzidube which, together with hydrogen peroxide, results in a colored reaction. The intensity of the color is measured using a spectrophotometer at 450 nm in optical density units.

To measure a free form of the TAM receptor ligand, the plasma sample is first subjected to a polyethylene glycol precipitation to precipitate a protein complex comprising the ligand. The supernatant fluid then only comprises the free form.

### Example 2

### Determination of Free and Complexed Protein S

An ELISA kit is available from Helena Laboratories (Catalog No. 5292) for measuring free and complexed Protein S.

The Protein S ELISA kit is a sandwich ELISA. Capture antibody specific for human Protein S is coated to 96-microwell polystyrene plates. Diluted patient plasma is incubated in the wells to allow Protein S to bind to the antibody. Bound Protein S is quantified using horseradish peroxidase conjugated anti-human Protein S detection antibody. A chromogenic substrate of tetramethylbenzidube and hydrogen peroxide is added to develop a colored reaction. The intensity of the color is measured using a spectrophotometer at 450 nm in optical density units. Protein S concentration in patient plasma is determined against a curve prepared from a reference plasma provided with the kit and is expressed as “percentage normal Protein S”. Results obtained from diluted plasma samples not pretreated with polyethylene glycol represent total Protein S concentration.

To measure free Protein S, polyethylene glycol is added to plasma samples prior to beginning the assay to precipitate the Protein S-C4b binding protein complex. The supernatant fraction containing free Protein S is then used along with the untreated plasma samples and free Protein S concentrations are determined using the same procedure as above using separate reference curves.

The levels of total Protein S (free+complexed) and free Protein S are expressed as a percentage of normal Protein S levels.

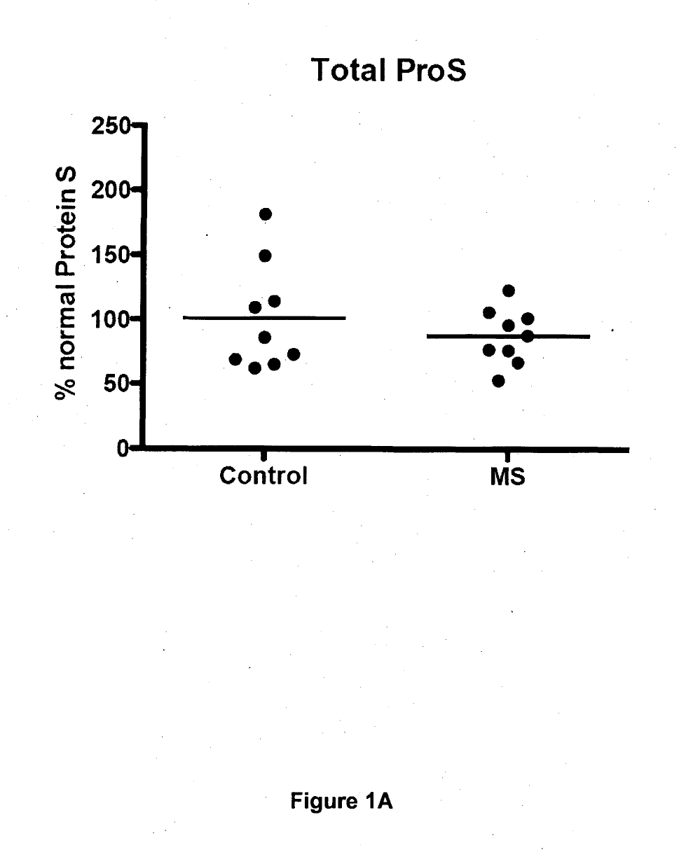

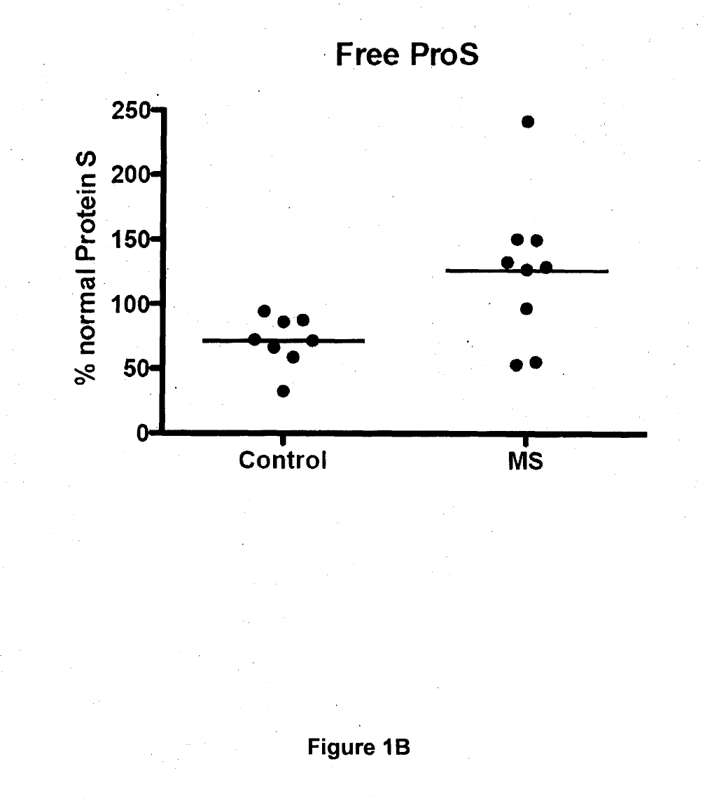

The results are shown in FIGS. 1A and 1B. The results clearly show that Protein S levels are elevated in the plasma of subjects with MS. This indicates that Protein S is an indicator of development of MS in a subject.

### Example 3

### Generation and Testing of Recombinant GAS6 and Protein S

cDNAs encoding each of GAS6 and Protein S are separately cloned into a baculovirus vector for expression and purification of recombinant protein in insect cells. The pIB/V5-HISTOPOTA expression kit (Invitrogen) is a suitable vector. Recombinant protein once purified is tested in an oligodendrocyte survival assay. Antibodies to naturally occurring GAS6 and Protein S are also tested to ascertain their binding ability to the recombinant forms.

### Example 4

### Generation of Triple Knock-Out Cells Transduced with TAM Receptor

A murine embryonic cell line (MEF) cell line is derived from triple TAM knockout mice and has been immortalized with the SV40 large T antigen. Replication incompetent retroviral vectors are generated which incorporate expression constructs of each of the TAM receptors. The MEF cells are transduced with various combinations of the TAM expressing clones. Expression of RNA encoding the relevant TAM receptor(s) is confirmed in each of the cell lines by quantitative PCR and the presence of the relevant proteins is confirmed by Western blot analysis utilizing commercially available polyclonal antibodies. Once the clones are constructed and the expression of the relevant receptors confirmed, the capacity for TAM ligands (GAS6) to induce downstream signaling events, in particular Akt and STAT1 phosphorylation is assessed by Western blot analysis.

### Example 5

### Small Molecule Screening Assay

The screening of small molecule agonists of TAM receptor signaling is based on the induction of two key downstream signaling events:

a) the induction of phosphorylation of an Akt substrate; and

b) the induction of phosphorylation of STAT1.

The rationale for adopting these complementary approaches is that:

a) that activation of the P13K/Akt pathway is known to occur in the context of important regulatory events such as the potentiation of oligodendrocyte survival and the modulation of innate immunity; and

b) there is direct molecular evidence to indicate that activation of sTAT1 is dependant on co-localization of the Axl and interferon A receptor (IFNAR) and that this is a direct mechanism by which a pro-inflammatory innate immune response is inhibited.

A mammalian expression vector for GFP-PRAS40 (PRAS40 representing a known Akt1 substrate) is generated by Gateway cloning technology utilizing a lentiviral destination vector according to standard protocols. All DNA constructs are validated by sequencing. DNA transfection is undertaken according to standard protocols into the various TAM-transduced fibroblast lines generated in Example 4. Clonal population of stable transfectants are utilized to facilitate equivalent levels of expression of the GST fusion protein at baseline. To enable this, cell lines expressing GFP-PRAS40 are subject to antibiotic selection and clonal populations are generated with the aid of FACS using GFP fluorescence as a sorting marker. Following clonal expansion, individual clones are screening for GAS6 responsiveness utilizing the LanthaScreen (Carlson et al. (2009) Journal of Biomolecular Screening 14:121-132) and in particular the kinetics of PRAS40 phosphorylation is determined. In addition, validation is undertaken via Western blot, utilizing standard protocols.

A similar approach is adopted for the STAT1 work in which the mammalian expression vector GFP-STAT1 is generated. In this instance the kinetics of phosphorylation of STAT1 in the transfected cell lines is assessed utilizing antibodies against Y701 whose phosphorylation is required for STAT1 transcriptional activation.

The assay for small molecule agonists is undertaken utilizing 384-well, flat-bottom, polystyrene tissue culture-treated microplates.

The assay is optimized by taking account of the agonist stimulation time, the number of cells plated per well and assay equilibrium time. Optimal buffer composition and antibody concentration are also assessed.

For phosphoprotein analysis, cells are lysed, cocktails for protease and phosphatase inhibitors added and Tb-labeled phosphorylation site-specific antibody is added. The assay plate is then allowed to equilibrate at RT and TB-FRET values are measured in a BMG fluorescence plate reader. Three replicates for each condition are undertaken and a soe-response curve is established with TB-FRET emission ratios normalized against unstimulated values. Validation of positive results is undertaken utilizing anti-GFP Western blot and by establishing the expected sensitivity and pharmacology to known P13K/AKT and STAT1 inhibitors. Treatment of the cells with agonists known not to activate P13K/AKT or STAT1 is also undertaken as controls.

Those skilled in the art will appreciate that aspects of the subject matter described. It is to be understood that the disclosure encompasses all such variations and modifications. The disclosure also includes all of the steps, features, compositions and compounds referred to or indicated in this specification, individually or collectively, and any and all combinations of any two or more of the steps or features.

### BIBLIOGRAPHY

Barnett and Prineas (2004) Ann. Neurol 55:459-468
Biggs et al. (1999) Cytometry 36:36-45
Binder et al. (2008) The Journal of Neuroscience 28(2):5195-5206
Carlson et al. (2009) Journal of Biomolecular Screening 14:121-132
Daneshvar et al. (1999) J. Immunol. Methods 226(1-2):119-128
Durig et al. (1993) J. Raman Spectrosc. 24(5):281-285
Eriksson et al. (1993) Biophys. J. 2:64
Fu et al. (1999) Nature Biotechnology 17:1109-1111, 1999
Johnson et al. (1993) Peptide Turn Mimetics in Biotechnology and Pharmacy, Pezzuto et al (Eds), Chapman and Hall, New York
Lai and Lemke (1991) Neuron 6:691-704
Lakowicz et al. (1997) Biophys. J. 72:567
Lemke and Rothlin (2008) Nature Reviews (Immunology) 8:327-336
Lewis et al. (1999) Dyes Pigm. 42(2):197
Malemed et al. (1990) “Flow cytometry and sorting”, 2^{nd} Ed., New York, Wiley-Liss
Petkovic-Duran et al. (2009) Biotechniques 47:827-834
Rahman et al. (1998) J. Org. Chem. 63:6196
Rapaport et al. (1999) Appl. Phys. Lett. 74(3):329-331
Tawa et al., Mater. Res. Soc. Symp. Proc.488 [Electrical, Optical and Magnetic Properties of Organic Solid-State Materials IV], 885-890
Youvan et al. (1997) Biotechnology et elia 3:1-18

1. A method for detecting the presence of an inflammatory neuropathological disease or condition in a subject, said method comprising screening blood plasma or other suitable blood fluid from said subject for a TAM receptor ligand or metabolite or precursor thereof wherein an altered level of the ligand or its metabolite or precursor relative to a control is indicative of the presence of an inflammatory neuropathological disease or condition or a likelihood of developing same.

2. The method of claim 1 wherein the TAM receptor ligand is free ligand.

3. The method of claim 1 wherein the TAM receptor ligand is Protein S or GAS6 or a metabolite or precursor thereof.

4. The method of claim 3 wherein the TAM receptor ligand is free Protein S.

5. The method of claim 1 wherein the subject is a human.

6. The method of claim 5 wherein the level of ligand is elevated.

7. The method of claim 5 wherein the inflammatory neuropathological disease or condition is selected from the group consisting of multiple sclerosis (MS), oligodendrocyte disease, acute disseminated encephalomyelitis, optic neuropathy (including neuromyelitis optic with transient autonomic disturbances), Devic's neuromyelitis optica, tropical spastic paraparesis, non-compressive myelopathies, concentric sclerosis, diffuse sclerosis acute hemorrhagic leukoencephalopathy, metachromatic leukodystrophy, leucoareosis, acute discriminated encephalomyelitis, progressive multifocal leukoencephalopathy, multisystem entrophy and brain trauma resulting in white matter.

8. The method of claim 7 wherein the inflammatory neuropathological condition or disease is MS, oligodendrocyte disease, stroke or brain trauma.

9. The method of claim 1 wherein the TAM receptor ligand or is metabolite or precursor is detected by an immunoassay.

10. The method of claim 9 wherein the immunoassay is a sandwich ELISA.

11. The method of claim 10 wherein blood plasma or other suitable is contacted with an immobilized antibody or antigen-binding fragment thereof specific for the TAM receptor ligand or its metabolite or precursor for a time and under conditions sufficient for the antibody to capture the ligand and then identifying captured ligand by contacting the captured ligand with an antibody or antigen-binding fragment labeled with a reporter molecule or component of a signal generating pathway or reaction and then detecting the reporter molecule or generated signal.

12. The method of claim 11 wherein the second antibody or antigen-binding fragment is labeled with an enzyme.

13-23. (canceled)

24. The method of claim 31, wherein a level of the ligand or metabolite or precursor thereof greater than 105% of the level in a control is indicative of the presence of the inflammatory neuropathological disease or condition or a likelihood of developing same.

25. The method of claim 24, wherein the TAM receptor ligand is selected from the group consisting of Protein S, GAS6 and a metabolite or precursor thereof.

26. The method of claim 24, wherein the TAM receptor ligand is selected from the group consisting of Protein S and a metabolite or precursor thereof.

27. The method of claim 31, wherein a level of the ligand or metabolite or precursor thereof 20% to 95% of the levels in a control is indicative of the presence of the inflammatory neuropathological disease or condition or a likelihood of developing same.

28. The A method of claim 27, wherein the TAM receptor ligand is selected from the group consisting of Protein S, GAS6 and a metabolite or precursor thereof.

29. The method of claim 27, wherein the TAM receptor ligand is selected from the group consisting of Protein S and a metabolite or precursor thereof.

30. A method for monitoring progression of an inflammatory neuropathological disease or condition in a subject, the method comprising screening blood plasma or other suitable blood fluid in said subject for a TAM receptor ligand or a metabolite or precursor thereof over time wherein a change in levels of ligand within a time period is indicative of disease progression.

31. A method for detecting the presence of an inflammatory neuropathological condition or disease or monitoring its progression in a subject, the method comprising screening blood plasma or other suitable blood fluid from said subject for levels of a TAM receptor ligand or a metabolite or precursor thereof, wherein an elevated level of the ligand or a metabolite or precursor thereof compared to a control is indicative of the presence of an inflammatory neuropathological disease or condition or a likelihood of developing same or the continued progression of the disease or condition.

32. A method of treating, controlling or preventing development of an inflammatory neuropathological disease or condition, the method comprising measuring the level of a TAM receptor ligand in a subject and comparing the level to a control, the control being a level from a normal, healthy subject or population of subjects or a level from the same subject as being treated taken at an earlier time point, and if the level of the TAM receptor ligand is altered compared to the control administering to the subject an effective amount of an agent selected from the group consisting of:
(i) interferon β or another immunomodulatory agent which influences the clinical course of the inflammatory neuropathological disease or condition;
(ii) an agent which reduces demyelination leading to a reduction in plasma levels of the TAM receptor ligand; and
(iii) an agent which normalizes levels of the TAM receptor ligand while inducing or exacerbating inflammatory neuropathology.
